(19) United States
(12) Patent Application Publication
(10) Pub. No.: US 2010/0286201 A1
(43) Pub. Date: Nov. 11, 2010

(54) POLYMORPH FORMS OF (S)-2-((4-BENZOFURANYL)CARBONYLAMINOMETHYL)-1-((4-(2-METHYL-5-(4-FLUOROPHENYL)THIAZOLYL)CARBONYL)PIPERIDINE

(76) Inventor: Leanda Jane Kindon, King of Prussia, PA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/677,614

(22) PCT Filed: Sep. 11, 2008
(86) PCT No.: PCT/EP08/62064

§ 371 Date: May 6, 2010

(30) Foreign Application Priority Data
Sep. 13, 2007 (GB) 0717882.5

(51) Int. Cl. A61K 31/454; C07D 417/14

(52) U.S. Cl. 514/320; 546/196

(57) ABSTRACT

New polymorphic forms of (S)-2-((4-benzofuranyl)carbonylamino methyl)-1-((4-(2-methyl-5-(4-fluorophenyl))thiazolyl)carbonyl)piperidine, methods for their preparation and use in medicine.

This invention relates to a novel pharmaceutical, to a process for the preparation of the pharmaceutical and to the use of the pharmaceutical in medicine.

International Patent Application, Publication Number WO 01/96302 discloses certain N-aroyl cyclic amine derivatives including (S)-2-((4-benzofuranyl)carbonylamino methyl)-1-((4-(2-methyl-5-(4-fluorophenyl))thiazolyl)carbonyl)piperidine.

It has now been discovered that (S)-2-((4-benzofuranyl)carbonylamino methyl)-1-((4-(2-methyl-5-(4-fluorophenyl))thiazolyl)carbonyl)piperidine exists in a series of novel polymorphic forms.

The series of novel polymorphic forms (hereinafter referred to as Form 1, Form 2, Form 3 and Solvated Form) have useful pharmaceutical properties and in particular they are indicated to be useful for the treatment and/or prophylaxis of diseases and disorders including, but not limited to, obesity and sleep disorders such as insomnia.

Accordingly, the present invention provides a polymorphic form of (S)-2-((4-benzofuranyl)carbonylamino methyl)-1-((4-(2-methyl-5-(4-fluorophenyl))thiazolyl)carbonyl)piperidine (Form 1) characterised in that it:

i) provides X-ray powder diffraction (XRPD) diffractogram comprising the following peaks:

| Diffraction angle | Lattice spacing |
|---|---|
| (°2θ) | (Å) |
| 9.1 | 9.7 |
| 18.9 | 4.7; |

and/or
ii) has an onset of melting typically in the range 163-173° C., as measured by DSC.

In a further aspect, the Form 1 polymorph provides an X-ray powder diffraction (XRPD) diffractogram comprising the following peaks:

| Diffraction angle | Lattice spacing |
|---|---|
| (°2θ) | (Å) |
| 8.0 | 11.1 |
| 9.1 | 9.7 |
| 13.4 | 6.6 |
| 14.1 | 6.3 |
| 18.9 | 4.7 |
| 20.2 | 4.4 |
| 23.5 | 3.8 |
| 30.6 | 2.9 |

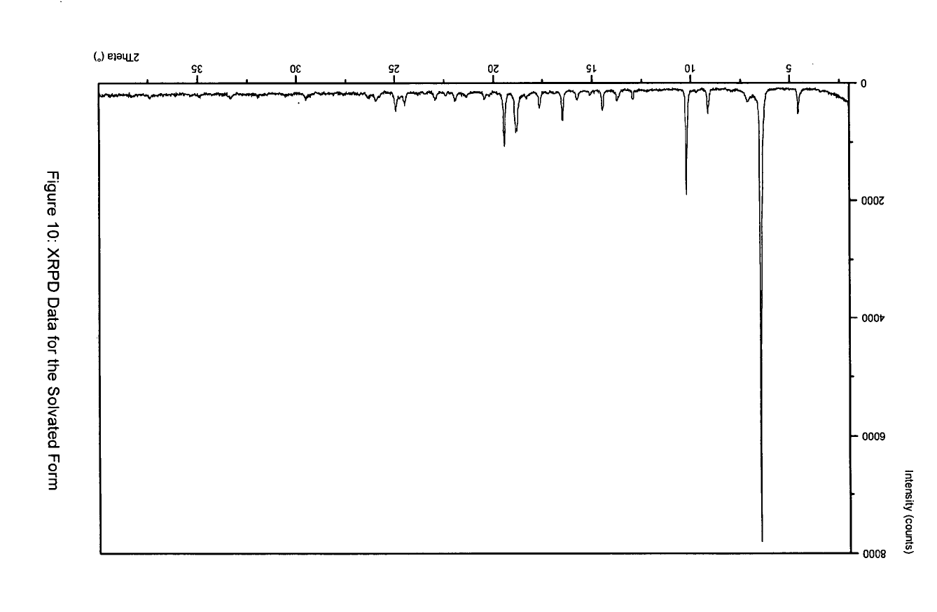

In a yet further aspect, the Form 1 polymorph provides an X-ray powder diffraction (XRPD) pattern substantially in accordance with FIG. 7.

It will be appreciated that references herein to “substantially” should be interpreted as encompassing a pattern within acceptable boundaries of experimentation.

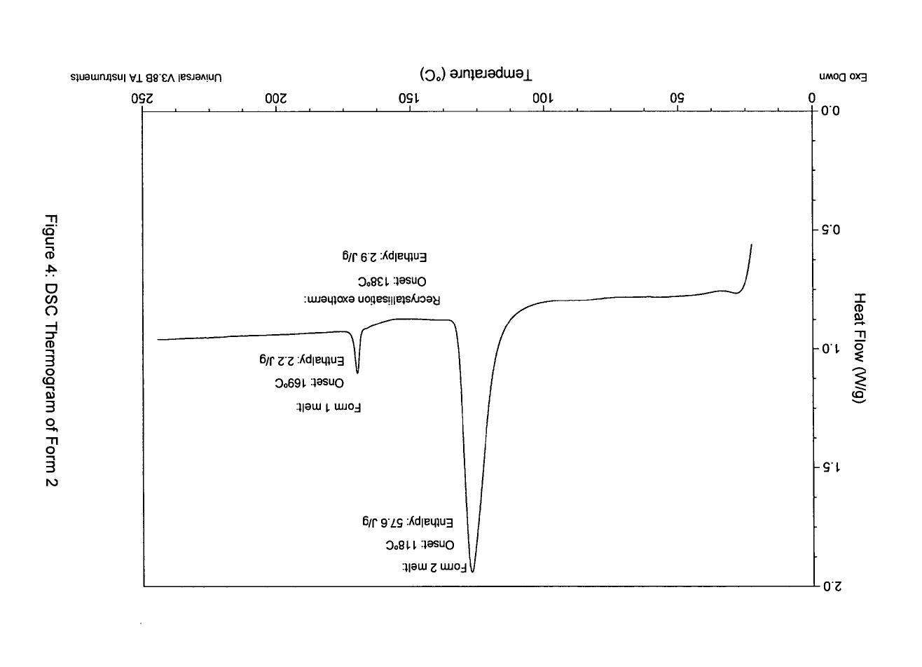

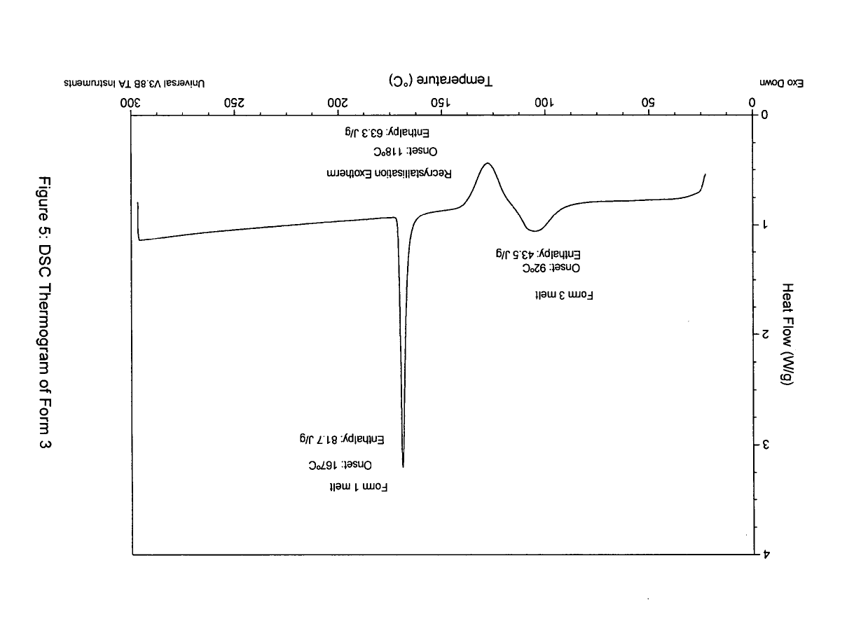

In a more particular aspect, the Form 1 polymorph provides a DSC thermogram substantially in accordance with FIG. 3.

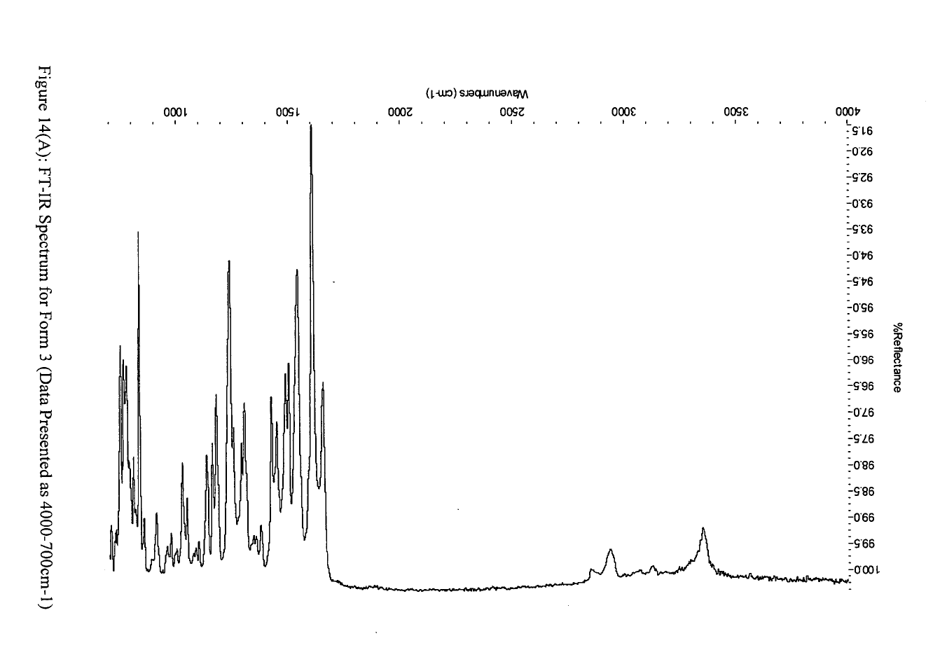

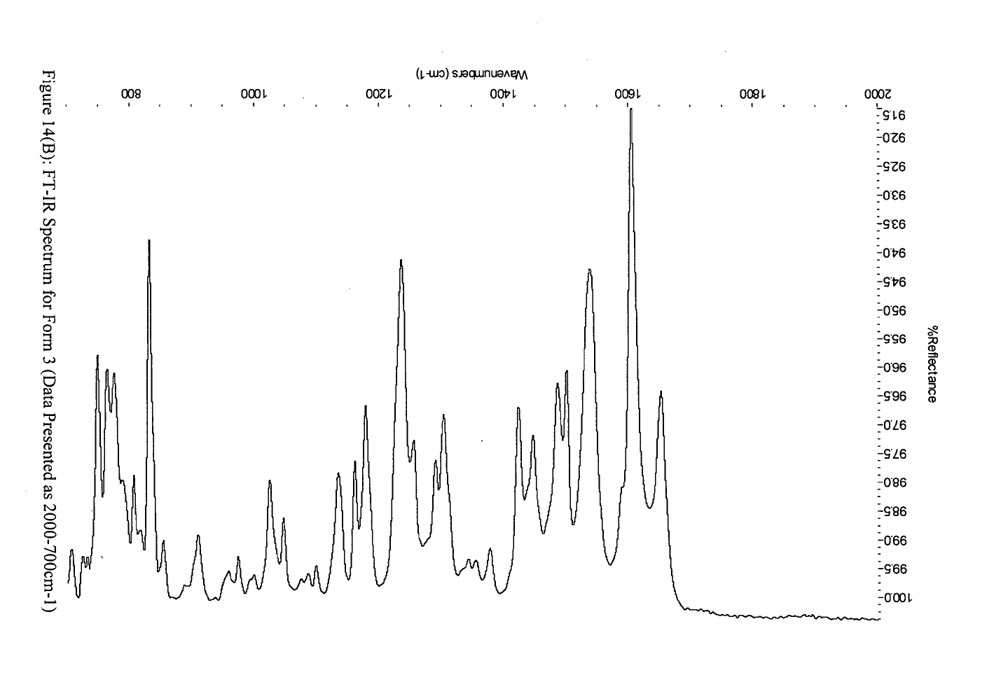

In a further aspect, the Form 1 polymorph provides an FT-IR spectrum substantially in accordance with FIG. 12.

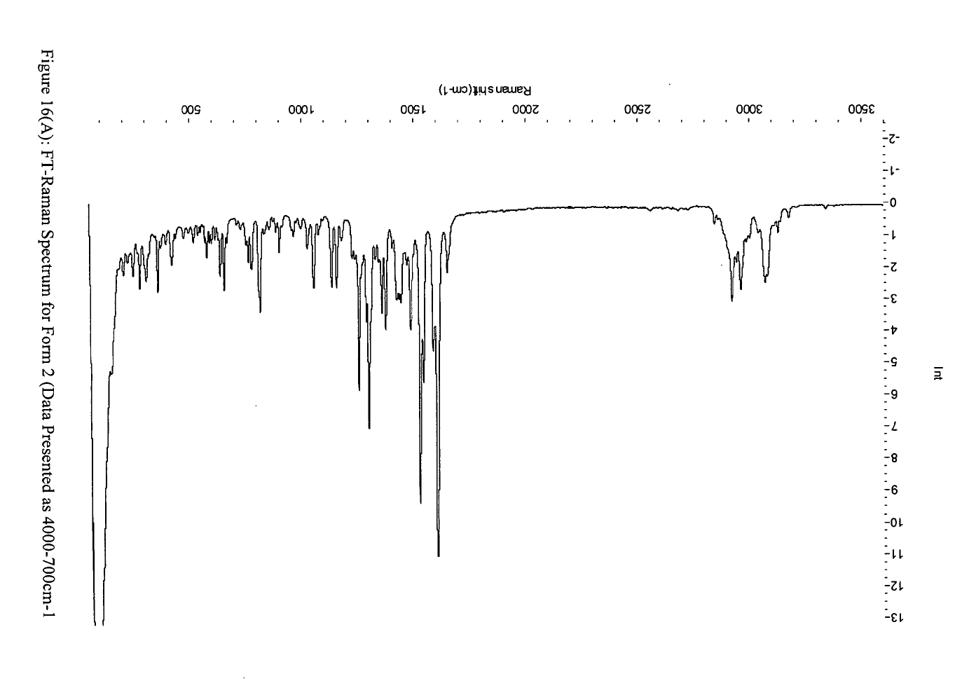

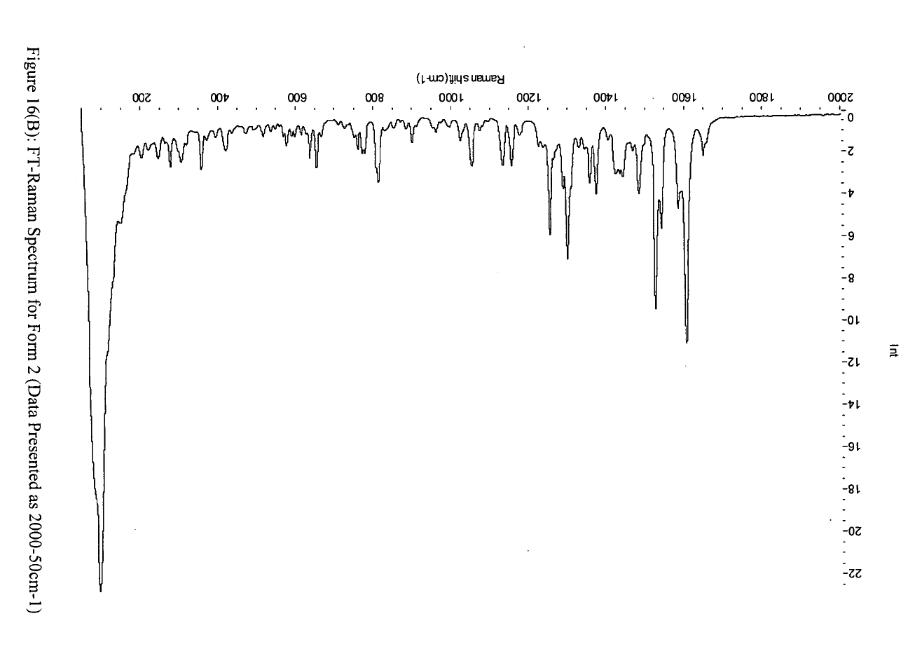

In a further aspect, the Form 1 polymorph provides an FT-Raman spectrum substantially in accordance with FIG. 15.

In a further embodiment the present invention also provides a polymorphic form of (S)-2-((4-benzofuranyl)carbonylamino methyl)-1-((4-(2-methyl-5-(4-fluorophenyl))thiazolyl)carbonyl)piperidine (Form 2) characterised in that it:

i) provides X-ray powder diffraction (XRPD) diffractogram comprising the following peaks:

| Diffraction angle | Lattice spacing |
|---|---|
| (°2θ) | (Å) |
| 9.9 | 9.0 |
| 15.8 | 5.6; |

and/or
ii) has an onset of melting typically in the range 113-123° C., as measured by DSC.

In a further aspect, the Form 2 polymorph provides an X-ray powder diffraction (XRPD) diffractogram comprising the following peaks:

| Diffraction angle | Lattice spacing |
|---|---|
| (°2θ) | (Å) |
| 7.6 | 11.7 |
| 9.9 | 9.0 |
| 11.1 | 8.0 |
| 13.1 | 6.8 |
| 15.8 | 5.6 |
| 17.8 | 5.0 |

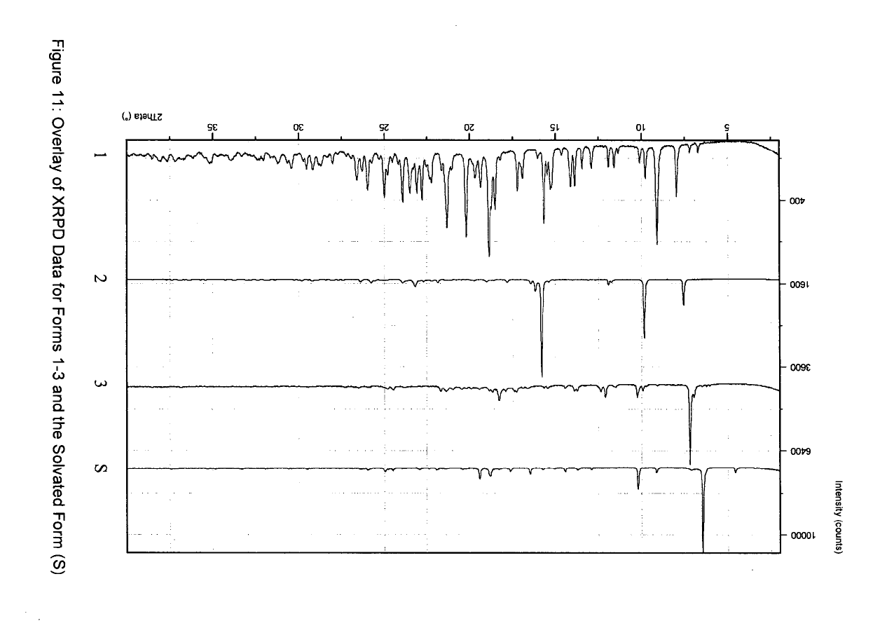

More particularly, the Form 2 polymorph provides an X-ray powder diffraction (XRPD) pattern substantially in accordance with FIG. 8.

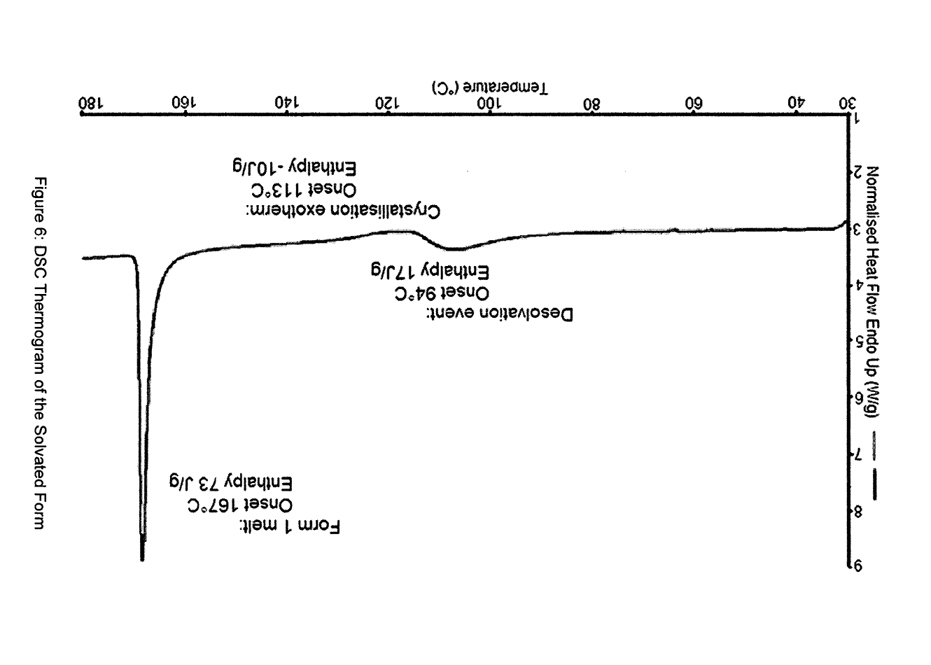

In a more particular aspect, the Form 2 polymorph provides a DSC thermogram substantially in accordance with FIG. 4.

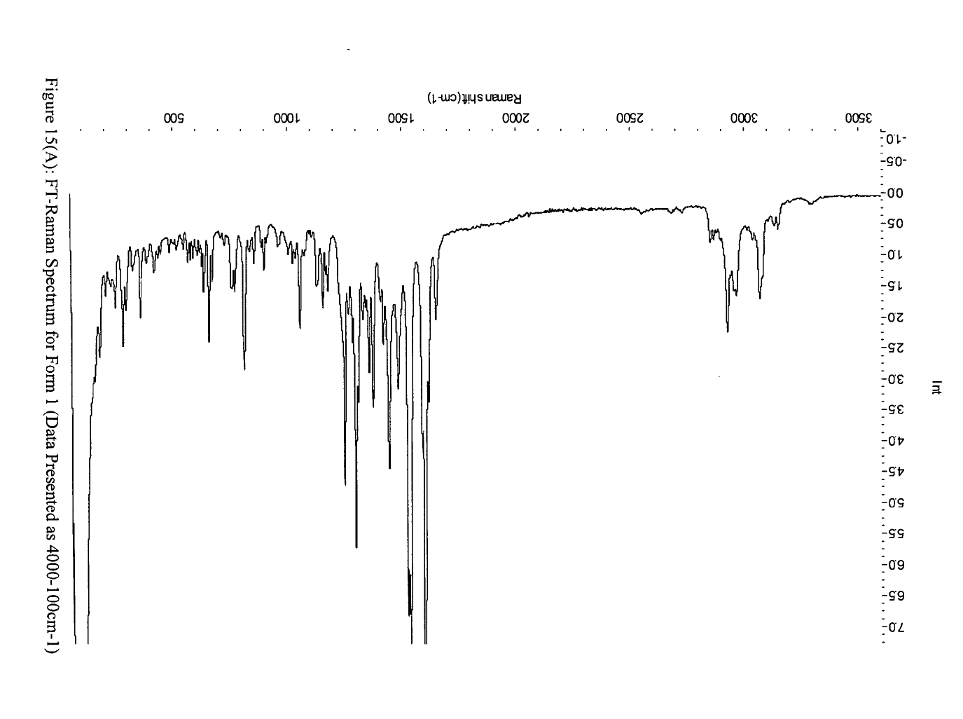

In a further aspect, the Form 2 polymorph provides an FT-IR spectrum substantially in accordance with FIG. 13.

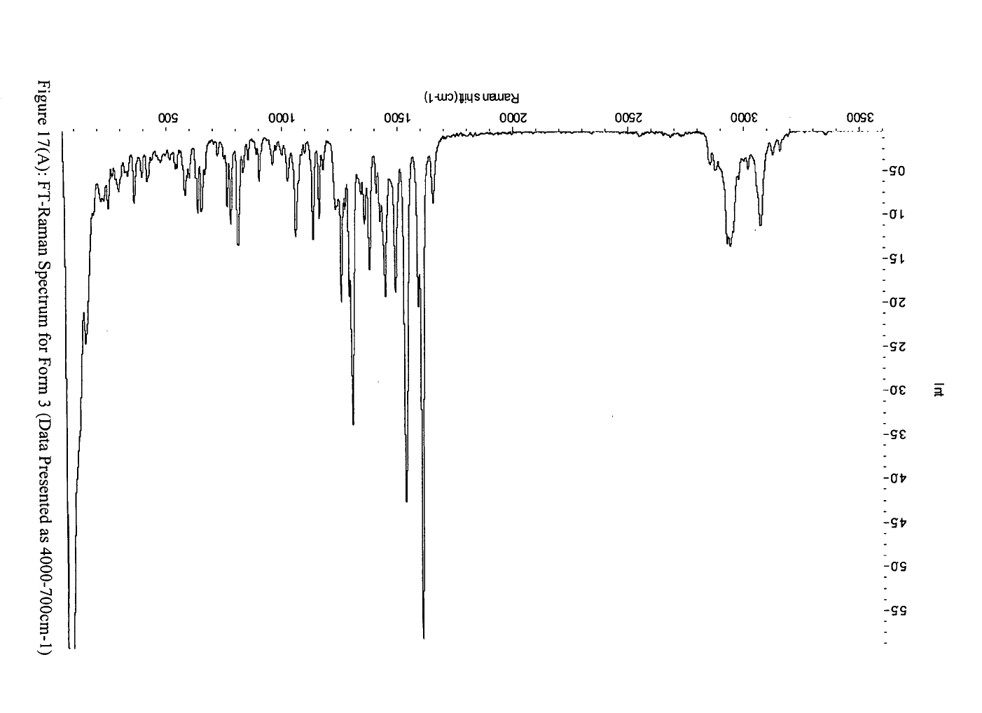

In a further aspect, the Form 2 polymorph provides an FT-Raman spectrum substantially in accordance with FIG. 16.

In a further embodiment the present invention also provides a polymorphic form of (S)-2-((4-benzofuranyl)carbonylamino methyl)-1-((4-(2-methyl-5-(4-fluorophenyl))thiazolyl)carbonyl)piperidine (Form 3) characterised in that it:

i) provides X-ray powder diffraction (XRPD) diffractogram comprising the following peaks:

| Diffraction angle | Lattice spacing |
|---|---|
| (°2θ) | (Å) |
| 7.0 | 12.7 |
| 18.3 | 4.8; |

and/or
ii) has an onset of melting typically in the range 87-97° C., as measured by DSC.

In a further aspect, the Form 3 polymorph provides an X-ray powder diffraction (XRPD) diffractogram comprising the following peaks:

| Diffraction angle | Lattice spacing |
|---|---|
| (°2θ) | (Å) |
| 6.1 | 14.6 |
| 6.2 | 14.2 |
| 7.0 | 12.7 |
| 12.4 | 7.2 |
| 18.3 | 4.8 |

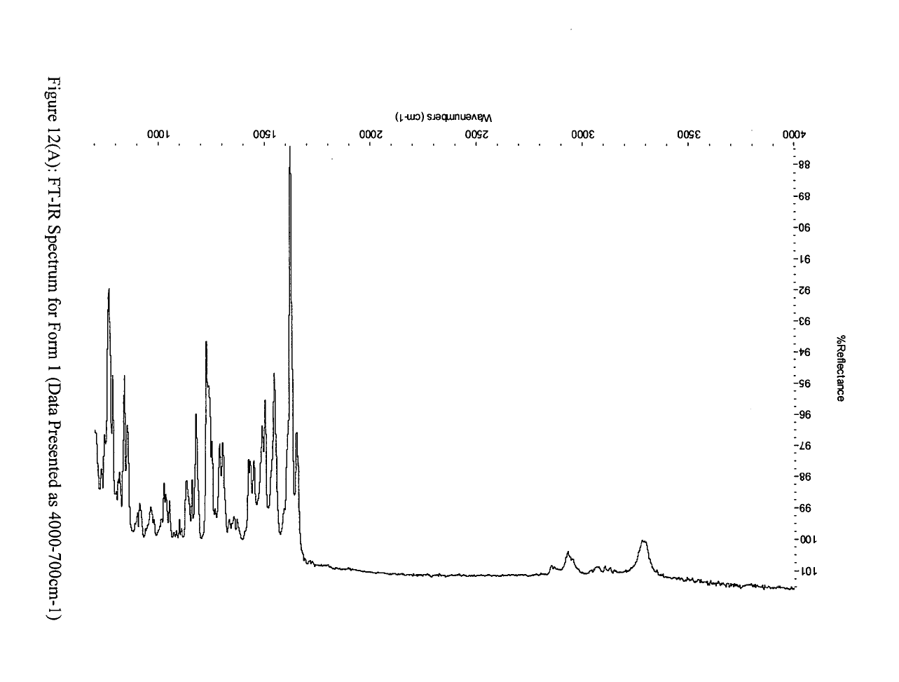

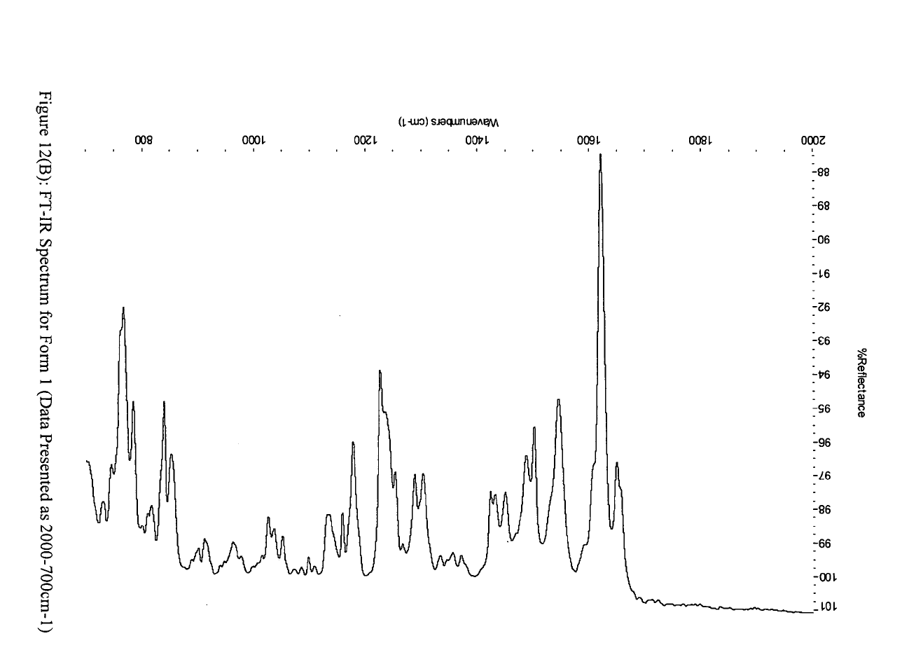

More particularly, the Form 3 polymorph provides an X-ray powder diffraction (XRPD) pattern substantially in accordance with FIG. 9.

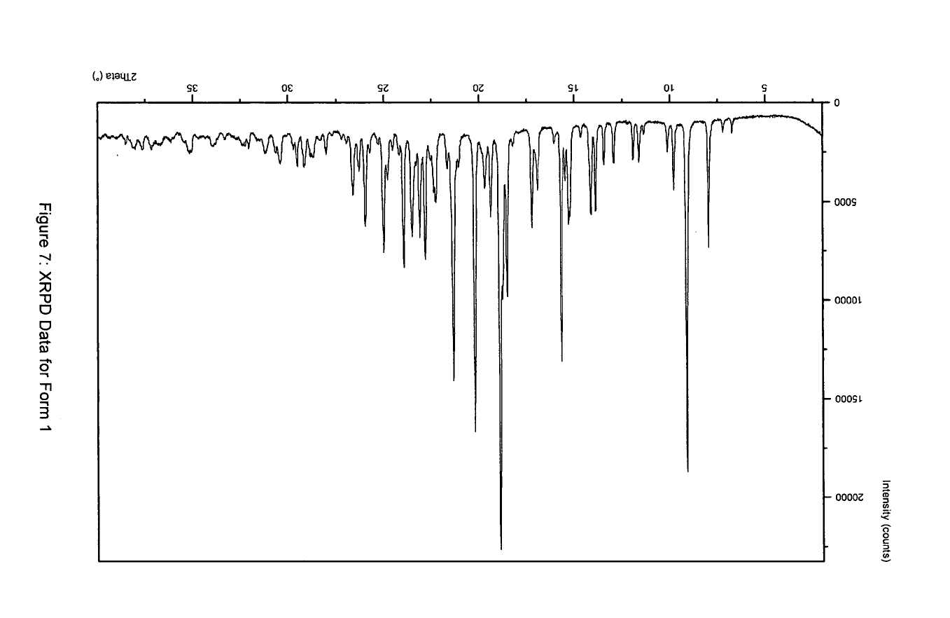

In a more particular aspect, the Form 3 polymorph provides a DSC thermogram substantially in accordance with FIG. 5.

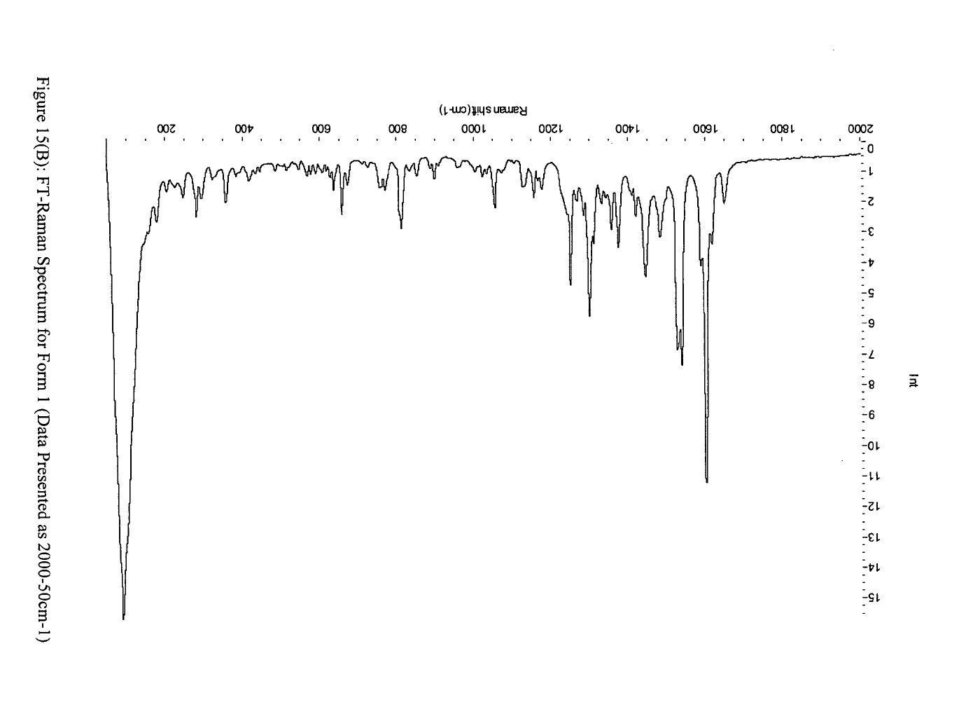

In a further aspect, the Form 3 polymorph provides an FT-IR spectrum substantially in accordance with FIG. 14.

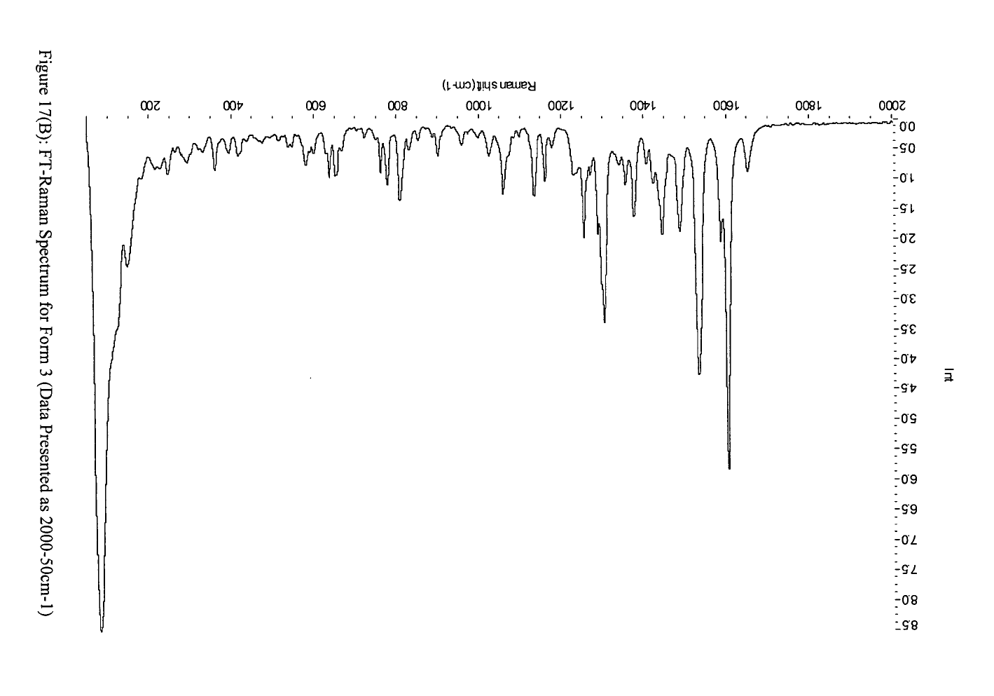

In a further aspect, the Form 3 polymorph provides an FT-Raman spectrum substantially in accordance with FIG. 17.

In a further embodiment the present invention also provides a polymorphic form of (S)-2-((4-benzofuranyl)carbonylamino methyl)-1-((4-(2-methyl-5-(4-fluorophenyl))thiazolyl)carbonyl)piperidine (the Solvated Form) characterised in that it:

i) provides X-ray powder diffraction (XRPD) diffractogram comprising the following peaks:

| Diffraction angle | Lattice spacing |
|---|---|
| (°2θ) | (Å) |
| 4.6 | 19.3; |

and/or
ii) has an onset of desolvation typically in the range 89-99° C., as measured by DSC.

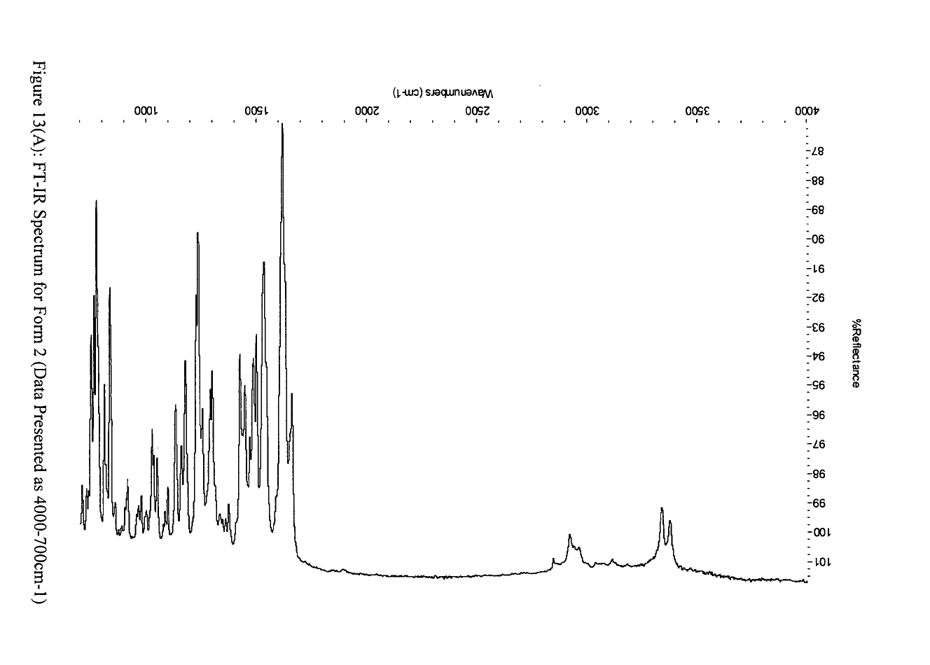

More particularly, the Solvated Form polymorph provides an X-ray powder diffraction (XRPD) pattern substantially in accordance with FIG. 10.

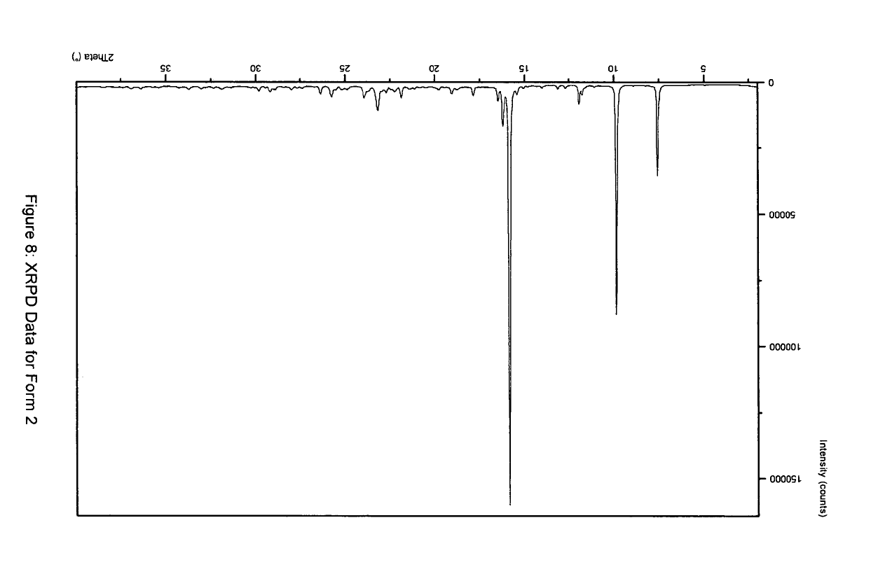

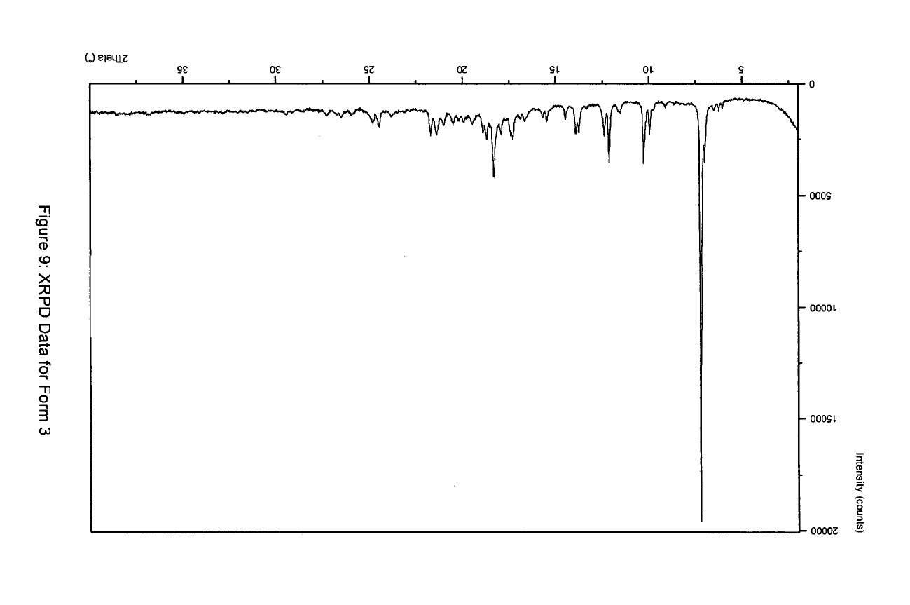

In a more particular aspect, the Solvated Form polymorph provides a DSC thermogram substantially in accordance with FIG. 6.

The XRPD, DSC, FT-IR and FT-Raman characterising data mentioned above is collected as described more fully in the section entitled “Characterising Data”.

The present invention encompasses the polymorphs isolated in pure form or when admixed with other materials, for example other salts or solvates (inclusive of their polymorphs) of (S)-2-((4-benzofuranyl)carbonylamino methyl)-1-((4-(2-methyl-5-(4-fluorophenyl))thiazolyl)carbonyl)piperidine, or any other material.

Thus in one aspect there is provided the polymorphs in isolated or pure form. “Isolated” or “pure” form refers to a sample in which the polymorphs are present in an amount of >75%, particularly >90%, more particularly >95% and even more particularly >99% relative to other compounds or polymorphs of (S)-2-((4-benzofuranyl)carbonylamino methyl)-1-((4-(2-methyl-5-(4-fluorophenyl))thiazolyl)carbonyl)piperidine which may be present in the sample.

The invention also provides a process for preparing the polymorphs, characterised in that a suspension of (S)-2-((4-benzofuranyl)carbonylamino methyl)-1-((4-(2-methyl-5-(4-fluorophenyl))thiazolyl)carbonyl)piperidine in isopropanol is heated to 82° C. followed by crystallisation upon cooling. In the above-mentioned process the solution may be seeded with the polymorph once obtained to induce crystallisation but this is not essential.

(S)-2-((4-benzofuranyl)carbonylamino methyl)-1-((4-(2-methyl-5-(4-fluorophenyl))thiazolyl)carbonyl)piperidine may be prepared according to known procedures, such as those disclosed in WO 01/96302. The disclosure of WO 01/96302 is incorporated herein by reference.

As mentioned above the polymorphs of the invention have useful therapeutic properties. More particularly, the polymorphs of the present invention are believed to be of potential use in the treatment or prophylaxis of diseases or disorders where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the polymorphs of the present invention may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the polymorphs of the present invention may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the polymorphs of the present invention may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the polymorphs of the present invention may be of use for the treatment or prophylaxis of a disease or disorder where an antagonist of a human orexin receptor is required such as feeding disorders such as bulimia nervosa, binge eating, obesity, including obesity observed in Type 2 (non-insulin-dependent) diabetes patients. Further, the polymorphs of the invention may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

Accordingly, in one embodiment, the present invention provides a polymorph of the invention for use as a therapeutic substance. More particularly, the invention provides a polymorph of the invention for use as a therapeutic substance in the treatment or prophylaxis of sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In a further embodiment the invention provides a polymorph of the invention for use as a therapeutic substance in the treatment or prophylaxis of diseases or disorders selected from the group consisting of depression and mood disorders, anxiety, substance-related disorders and feeding disorders.

The invention further provides a method of treatment or prophylaxis of sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome, comprising administering an effective amount of the polymorph of the invention.

In a further embodiment the invention further provides a method of treatment or prophylaxis of diseases or disorders selected from the group consisting of depression and mood disorders, anxiety, substance-related disorders and feeding disorders, comprising administering an effective amount of the polymorph of the invention.

In another embodiment, the invention provides the use of a polymorph of the invention in the manufacture of a medicament for use in the treatment or prophylaxis of sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In further embodiment, the invention provides the use of a polymorph of the invention in the manufacture of a medicament for use in the treatment or prophylaxis of diseases or disorders selected from the group consisting of depression and mood disorders, anxiety, substance-related disorders and feeding disorders.

When used in therapy, the polymorphs are usually formulated in a standard pharmaceutical composition. Such compositions can be prepared using standard procedures.

Thus, the present invention further provides a pharmaceutical composition for use in the treatment or prophylaxis of sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome, which comprises a polymorph of the invention and a pharmaceutically acceptable carrier.

In a further embodiment the invention provides a pharmaceutical composition for use in the treatment or prophylaxis of diseases or disorders selected from the group consisting of depression and mood disorders, anxiety, substance-related disorders and feeding disorders.

The present invention further provides a pharmaceutical composition which comprises a polymorph of the invention and a pharmaceutically acceptable carrier.

The polymorphs of the invention may be used in combination with other therapeutic agents. When the polymorphs are used in combination with other therapeutic agents, the compounds may be administered either sequentially or simultaneously by any convenient route.

The invention thus provides, in a further embodiment, a combination comprising a polymorph of the invention together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a polymorph of the invention is used in combination with a second therapeutic agent active against the same disease state the dose of the polymorph may differ from that when the polymorph is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral, buccal, sublingual, nasal, rectal or transdermal administration. The polymorphs of the invention which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the polymorph of the invention used in the treatment or prophylaxis of the abovementioned disorders or diseases will vary in the usual way with the particular disorder or disease being treated, the weight of the subject and other similar factors. However as a general rule the composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 0.05 mg to 500 mg, of the active material, depending on the method of administration. The composition may contain from 50 mg to 1000 mg, for example from 100 mg to 400 mg of the carrier, depending on the method of administration. The dose of the polymorph of the invention used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, and such unit doses may be administered more than once a day, for example two or three times a day so that the total daily dosage is in the range of about 0.01 to 100 mg/kg. Such therapy may extend for a number of weeks or months.

The following Examples illustrate the preparation of the polymorphs of the invention. The Descriptions 1 to 12 illustrate the preparation of intermediates used to make the polymorphs. Example 2 describes the preparation of Form 1. Example 5 describes the preparation of Form 2 and examples 7 and 8 describe alternative methods to prepare Form 2. Example 6 describes the preparation of Form 3 and examples 9 to 13 describe alternative methods to prepare Form 3. Example 4 describes the solvated form and examples 14 to 17 describe alternative methods to prepare the solvated form.

### Abbreviations

### DMF Dimethylformamide

### IPA Isopropylalcohol

iPrOAc Isopropylacetate

### MeOH Methanol

NaOMe Sodium methoxide

### NEt₃ Triethylamine

### TBME Tertiarybutylmethylether

### THF Tetrahydrofuran

The following preparations are suitable for preparing the polymorphs of the invention at, for example, 60 g scale, 600 g scale, 6 kg scale and 60 kg scale.

### Intermediate 1

### (3R,5S,8aR)-3-Phenylhexahydro-5H-[1,3]oxazolo[3,2-a]pyridine-5-carbonitrile

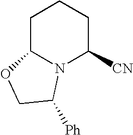

R-(−)-Phenylglycinol (1 wt, 1 eq), citric acid (8.4 wt, 6 eq.) and water (10 vol) were charged into the reactor. The solution was cooled to 0° C. and glutaric dialdehyde 25 wt. % solution in water (4.12 vol, 1.5 eq.) was added over 10 minutes. Stirring at 0° C. was continued for 20 minutes.

A solution of potassium cyanide (0.71 wt, 1.5 eq.) in water (2 vol) and dichloromethane (7 vol) was added over 10 minutes. The reaction mixture was warmed to 20° C. over 2 hours and stirred for 90 minutes.

The pH was adjusted to 8 by addition of NaOH 30 wt. % (12.8 vol), the layers were separated, and the mixture was extracted with dichloromethane (3×3 vol). The combined organic layers were dried with sodium sulfate (1 wt). Zinc bromide (0.2 wt, 0.12 eq.) was added and the mixture was stirred vigorously for 13 hours at reflux. The product mixture was filtered through silica gel (3 wt) and washed with dichloromethane (2×10 vol).

The organic layers were concentrated at 45° C. jacket temperature and reduced pressure to ˜10% of their original volume. Methanol (10.6 vol) was added and the distillation was continued. 10.6 vol of solvent mixture was removed. The amount of methanol was adjusted to 0.40 L per mol of the product. The mixture was heated to 45° C. and water (0.108 L per mol of the product) was added. The mixture was cooled to 43° C. After start of the crystallisation the suspension was cooled to 0° C. over 5 hours and stirred at this temperature for 1 hour. The product was collected by filtration and washed with a mixture of MeOH (0.15 L per mol of the product) and water (0.04 L per mol of the product) at 0° C. and dried at 40° C. jacket temperature and reduced pressure for 6 hours to yield the title compound as a white solid.

Yield (% theory): 63%

### Intermediate 2

### (2R)-2-[(2S)-2-(Aminomethyl)piperidin-1-yl]-2-phenylethanol

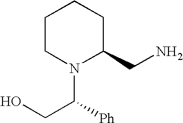

Intermediate 1 (1 wt, 1 eq) was dissolved in THF (9.2 vol) and the solution was cooled to −5° C. A solution of LiAlH₄ in THF (2.3 M, 3.8 vol, 2 eq) was added slowly at −5 to 5° C. After complete addition stirring was continued at 0° C. for 1 hour. The white suspension was heated to 37° C. for 16 hours.

The reaction mixture was cooled to −5° C. and quenched at −5 to 5° C. by careful addition of water (1.33 vol) then 30% NaOH (0.17 vol). THF (0.22 vol) was added during the quench. The suspension was heated to 20° C., sodium sulfate (4 wt) was added, and the mixture was stirred for 30 minutes. The salts were removed by filtration and the filter cake was washed with THF (2×3 vol).

Prior to use in the preparation of Intermediate 3, the solution of the title compound was concentrated to 10.25 vol.

### Intermediate 3

### tert-Butyl ({(2S)-1-[(1R)-2-hydroxy-1-phenylethyl]piperidin-2-yl}methyl)carbamate

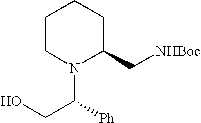

The weights and volumes refer to the amount of Intermediate 1.

At 0° C. a solution of di-tert-butyl dicarbonate (0.92 wt, 0.96 eq) in THF (1.84 vol) was added within 30 minutes to the solution of Intermediate 2 in THF. After complete addition the solution was warmed to 20° C. and stirred for 20 minutes. This solution of the title compound in THF was used directly in the next step.

### Intermediate 4

### tert-Butyl [(2S)-piperidin-2-ylmethyl]carbamate

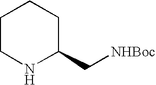

The weights and volumes refer to amount of Intermediate 1.

Palladium on charcoal (0.15 wt, 10% Pd/C, Johnson Matthey type 490 paste) and acetic acid (0.73 vol) were added to the solution of Intermediate 3 in THF under an atmosphere of nitrogen. Hydrogenation was done at 1200-1300 mbar and 40° C. Conversion was complete after 21 hours.

The catalyst was removed by filtration and the filter cake was washed with THF (2×2.2 vol). The combined filtrates were concentrated at 50° C. jacket temperature and reduced pressure to ˜2.8 vol. Toluene (2.6 vol) was added and the distillation was continued under reduced pressure to leave ˜2.8 vol.

Toluene (5 vol) was added and the product was extracted with an aqueous solution of citric acid (3×3 vol; citric acid: 450 g/L). The combined aqueous layers were extracted with toluene (4×5 vol). The pH of the aqueous layer was adjusted to >10 by addition of 30% NaOH (approx. 6.5 vol) and the mixture was extracted with THF (3×5 vol). The combined organic layers were dried with sodium sulfate (2 wt). The sodium sulfate was removed by filtration and the filter cake was washed with THF (2×1.3 vol).

The combined filtrates were concentrated at 50° C. jacket temperature and reduced pressure to 4.25 vol. Methyl cyclohexane (3×4.3 vol) was added in 3 portions and the distillation was continued. Further solvent (3×4.3 vol) was removed under reduced pressure.

Methyl cyclohexane (0.53 vol) was added, the suspension was cooled over 4 hours to −10° C., and stirred for 12 hours at this temperature. The slurry was filtered, washed with methyl cyclohexane (0.66 vol) at −10 to −5° C., and dried at 50° C. jacket temperature and reduced pressure to the title compound as a white solid.

Corrected yield (% theory): 87%

### Intermediate 5

### Methyl 3-chloro-3-(4-fluorophenyl)-2-oxopropanoate

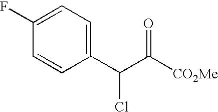

4-Fluorobenzaldehyde (1 wt, 1 eq), methyl dichloroacetate (1.07 vol, 1.3 eq) and TBME (5.1 vol) were charged into the reactor. NaOMe (0.55 wt, 1.25 eq) was continuously dosed over 2 hours at 20° C. The reaction mixture was heated to reflux for 1 hour. Water (2.0 vol) was added with vigorous stirring and the phases were separated. Solvent (3.3 vol) of the org. phase was removed at a jacket temperature of 60° C. under reduced pressure to give the title compound (3.3 vol) as a solution in TBME (57% w/w).

Yield (% theory): 102%

### Intermediate 6

### Methyl 5-(4-fluorophenyl)-2-methyl-1,3-thiazole-4-carboxylate

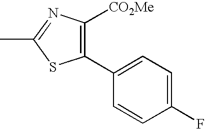

All volumes and weights refer to the corrected weight of Intermediate 5 in TBME solution.

Thioacetamide (0.23 wt, 0.7 eq, corrected for purity) in MeOH (2.4 vol) was heated to 64° C. A mixture of Intermediate 5 (1 wt, 1.0 eq) in TBME (58% w/w), 1.8 vol) was added over 30 minutes at 64-58° C., then stirring was continued for 1.5 hours at 58° C. Solvent (3.2 vol) was removed at a jacket temperature of 78° C. under reduced pressure. TBME (2.9 vol) was added and solvent (2.5 vol) was removed at a jacket temperature of 78° C. under reduced pressure. To the resulting slurry, TMBE (2.9 vol) was added and solvent (2.2 vol) was removed at a jacket temperature of 78° C. under reduced pressure. The resulting slurry was cooled to 5° C., stirred for 1 hour at 5° C. and filtered off to give the title compound (0.75 wt).

Yield (% theory): 57%

### Intermediate 7

### 5-(4-Fluorophenyl)-2-methyl-1,3-thiazole-4-carboxylic acid

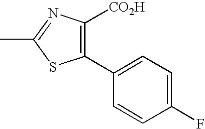

All volumes and weights refer to corrected weight of Intermediate 6.

The reactor was charged with Intermediate 6 (1 wt) and MeOH (4.8 vol). The solution was heated to 40° C. Aq. 3 N KOH (3.3 vol, 2.5 eq.) was added over 41 minutes at 40° C. Stirring was continued for 1.1 hours at 40° C. Solvent (4.8 vol) was removed at a jacket temperature of 60° C. under reduced pressure. IPA (0.9 vol) was added and the solution transferred into a dosing vessel. The reactor was charged with 5 N HCl (2.1 vol, 2.6 eq) and heated to 40° C. The solution of the K-salt in aq. KOH and IPA was added to the reactor at 40° C. over 23 minutes. Stirring was continued for 16 hours at 20-25° C. and 1 hour at 5° C. The suspension was filtered and the filter cake washed with water (7×1.3 vol) until a pH of 3 was reached. The product was dried on the rotavap (jacket=55° C., vac.) to give the title compound (0.58 wt) as a green solid.

Yield (% theory): 64%

### Intermediate 8

### Methyl 3-(allyloxy)benzoate

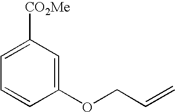

Methyl 3-hydroxybenzoate (1.0 wt), potassium carbonate (2.0 eq., 1.8 wt), acetone (6 vol) and allyl bromide (1.0 eq., 0.8 wt) were charged to the reactor. The feed vessel was rinsed two times with acetone (2×0.3 vol) and the mixture stirred at reflux for 5 hours. Additional allyl bromide (1.0 eq., 0.8 wt) was added, the feed vessel was rinsed two times with acetone (2×0.3 vol) and the mixture was stirred at reflux for 15.5 hours. The suspension was cooled to 20° C. and filtered, the filter cake was washed with acetone (2×1.0 vol). The filtrate was concentrated in the reactor at a jacket temperature of 50° C. Further concentration at the rotavap (50° C.) gave the title compound as a yellowish oil.

Yield (% theory): 100%

### Intermediate 9

### Methyl 2-allyl-3-hydroxybenzoate

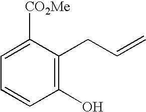

Intermediate 8 (0.2 wt.) was charged to the flask and heated to 230° C. The remainder of Intermediate 8 (0.8 wt.) was added at 230° C. over 42 minutes and the mixture was stirred at 230° C. for 63 minutes. After cooling to 36° C. the product was diluted with 0.9 vol TBME.

Further 1.1 vol TBME and 1.0 M LiOH (1.05 eq., 5.46 vol) were added at 36° C. and the mixture was stirred at 36° C. for 6 hours at 263 rpm. The mixture was cooled to 5° C. and 32% HCl (1.1 eq., 0.57 vol) was added to reach pH 1. The phases were separated and the aqueous phase was extracted with TBME (2×1 vol) at 20° C. The combined organic phases were extracted with saturated aqueous NaHCO₃-soln. (3×5 vol) and saturated aqueous NaCl-soln. (2 vol). The organic phase was concentrated to dryness on a rotavap (bath temperature 50° C.) to give the title compound as a brownish waxy solid.

Yield (% theory): 58%

### Intermediate 10

### Methyl 2-hydroxy-2,3-dihydro-1-benzofuran-4-carboxylate

A total of 10.48 kg Intermediate 9 was transformed in the ozonolysis step in three runs using a 40 L cryogenic reactor. The intermediates were combined and stored at −20° C. prior to the workup. The vacuum pump was running with a mixture of bleach and aq. NaOH.

The cryogenic reactor was charged with Intermediate 9 (1.0 wt.), MeOH (9.5 vol) and a saturated solution of Sudan Red 7B in methanol (0.65 mg/mL, 0.5 vol). Ozone was passed through the solution at −64 to −69° C. for 5 hours (flow of oxygen 1.2 m³/h, 1.2-1.6 kW) until the colour of the solution turned from intensive red to light brown. The solution was sparged with nitrogen at −70° C. for 5 minutes. Dimethylsulfide (4.0 eq., 1.5 vol) was added at −66 to −70° C. over 15 minutes and the mixture was warmed to 0° C. over 16 hours. A peroxide test (potassium iodide test strips) indicated absence of peroxide. The crude product solution was stored at −20° C.

The intermediate from the ozonolysis was concentrated at 50° C. jacket temperature and reduced pressure (5.2 vol solvent was distilled off), 7.8 vol toluene added, then 6.8 vol solvent was distilled off at 50° C. 7.8 vol toluene was added and 9.5 vol solvent was distilled off. The org. phase was washed with saturated aqueous NaCl-soln. (2×3 vol) at 20° C. to give the title compound as a yellow solution in toluene (15.1% w/v, 6.7 vol).

Yield (% theory): 99%

### Intermediate 11

### 1-Benzofuran-4-carboxylic acid

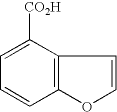

A solution of Intermediate 10 in toluene (1 wt in 6.6 vol) was added to a refluxing mixture of p-toluenesulfonic acid (0.03 eq., 0.03 wt) in toluene (3.8 vol) over 43 minutes. The mixture was stirred under reflux for 33 minutes then solvent (1.1 vol) was withdrawn at 130° C. jacket temperature under slightly reduced pressure (1000-880 mbar). The mixture was further stirred at reflux. 4.25 hours after the addition of Intermediate 10 the mixture was cooled to 25° C. The mixture was filtered over silica gel (1.1 wt) that had been conditioned with toluene (4.1 vol). The silica plug was further washed with toluene (8.2 vol). The product containing fractions were combined (total volume 10.8 vol). Solvent (8.6 vol) was withdrawn at 80° C. jacket temperature under reduced pressure. Toluene (0.6 vol) and 1.0 M NaOH (1.5 eq., 7.7 vol) was added and the two-phase mixture was stirred at 60° C. for 9 hours. After cooling to 20° C. the phases were separated and 32% HCl (1.7 vol) was added at 2-14° C. to the aqueous phase. After addition of 9.4 vol toluene the mixture was stirred under reflux for 14.5 hours. The suspension was cooled to 20° C. and THF (5.3 vol) was added. The phases were separated and the organic layer was washed two times with a mixture of 5.6 vol water and 0.6 vol THF. 10.3 vol of solvent was distilled off at 80° C. jacket temperature and reduced pressure before 3.2 vol toluene was added. The mixture was refluxed for 33 minutes (ambient pressure), cooled to 0° C. over 3 hours and stirred at that temperature for 2.5 days. The precipitate was filtered, dried for 2 hours in a stream of nitrogen and dried in a rotavap at 50° C. to give the title compound as an off-white solid.

Yield (% theory): 62%

### Intermediate 12

### tert-Butyl [((2S)-1-{[5-(4-fluorophenyl)-2-methyl-1,3-thiazol-4-yl]carbonyl}piperidin-2-yl)methyl]carbamate

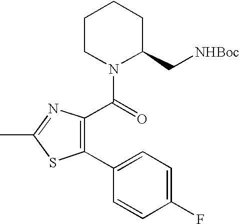

All volumes and weights refer to the weight of Intermediate 4.

The reactor was charged with Intermediate 7 (1.2 eq) and dichloromethane (8.6 vol). Solvent (6.9 vol) was removed at a jacket temperature of 46-49° C. under reduced pressure. Dichloromethane (8.6 vol) was added. Solvent (8.4 vol) was removed at a jacket temperature of 49° C. under reduced pressure. Dichloromethane (7.0 vol) was added. DMF (0.05 vol, 0.14 eq) and dichloromethane (0.2 vol) were added. A solution of oxalyl chloride (0.47 vol, 1.2 eq) in dichloromethane (0.2 vol) was added at 20-17° C. over 18 minutes. The addition vessel was rinsed with dichloromethane (0.2 vol) and the solution was added. The mixture was stirred for 2.5 hours at 16-21° C. The reaction mixture was stored under nitrogen in a feed tank, the reactor was rinsed with dichloromethane (1 vol) and this solution was added to the reaction mixture in the feed tank. The reactor was dried under vacuum and charged with Intermediate 4 (1.0 eq, 1.0 wt), dichloromethane (3.3 vol) and NEt₃ (2.0 vol, 3.0 eq). The solution in the feed tank was added at 20-24° C. over 48 minutes. The feed tank was rinsed with dichloromethane (0.3 vol) and the solution was added. The mixture was stirred overnight (13 hours) at 18-20° C.

20% aq K₂CO₃ (7 vol) was added at 20-17° C. over 18 minutes. The mixture was stirred for 1 hour at 17-21° C. After phase separation (2 hours) water (2.4 vol) was added to the lower organic phase at 18-19° C. over 1 minute. The mixture was stirred for 15 minutes at 19-20° C. After phase separation (42 min) solvent (11.6 vol) was removed at a max. jacket temperature of 49° C. under reduced pressure. Ethyl acetate (9.4 vol) was added. Solvent (9.4 vol) was removed at a maximum jacket temperature of 61° C. under reduced pressure. Ethyl acetate (9.4 vol) was added. Solvent (9.4 vol) was removed at a maximum jacket temperature of 79° C. under reduced pressure and heptane (10.7 vol) was added at 66-61° C. over 50 minutes. The mixture was cooled to 15° C. over 1 hour, stirred 15° C. overnight and filtered. The filter cake was washed with a mixture of ethyl acetate (0.29 vol) and heptane (2.1 vol) and dried in 3 portions at a maximum jacket temperature of 40° C. and reduced pressure at the rotavap to give the title compound as a beige solid.

Yield (% theory): 93%

### EXAMPLE 1

### Preparation of (S)-2-((4-benzofuranyl)carbonylamino methyl)-1-((4-(2-methyl-5-(4-fluorophenyl))thiazolyl)carbonyl)piperidine

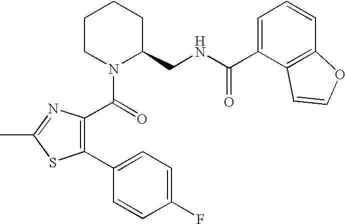

All volumes and weights refer to the weight of Intermediate 12.

The reactor was charged with Intermediate 11 (1.0 eq) and DMF (0.025 vol, 0.14 eq) in dichloromethane (1.9 vol) at 20° C. A solution of oxalyl chloride (0.20 vol, 1.0 eq) in dichloromethane (2.1 vol) was added at 20-19° C. over 30 minutes. The mixture was stirred 2 hours at 19-20° C. Solvent (2.8 vol) was removed at a maximum jacket temperature of 45° C. under reduced pressure. The solution was stored under nitrogen in a feed tank. The reactor was cleaned, dried under vacuum and charged with Intermediate 12 (1.0 eq, 1 wt) and dichloromethane (7.0 vol). Trifluoroacetic acid (3.05 wt, 12 eq) was added at 19-20° C. over 18 minutes. The mixture was stirred overnight at 20-21° C. The mixture was split in 2 equal portions. Each portion was washed with half saturated aqueous Na₂CO₃ (8.6 vol) at 20° C. The combined organic phases were dried over MgSO₄ (0.45 wt). After filtration the filtrate was transferred into the cleaned and dried reactor. Dichloromethane (1.3 vol) and triethylamine (0.96 vol, 3 eq) were added.

The acid chloride solution was added at 1-5° C. over 25 minutes and the mixture was stirred at 19-22° C. overnight. The mixture was split in 2 equal portions. Each portion was washed with saturated aqueous NaHCO₃ (7.3 vol) at 20° C. The combined organic phases were concentrated in the cleaned reactor. The mixture was filtered through a plug of silica gel (0.78 wt) conditioned with ethyl acetate and eluted with ethyl acetate (8.8 vol). The filtrate was concentrated in the cleaned reactor and a solvent change to iPrOAc was performed. The resulting suspension was heated to obtain a clear solution. The solution was cooled and seed crystals (obtained by cooling of about 1 vol % of the clear solution) was added at 57° C. The resulting suspension was concentrated, cooled to 10° C., stirred overnight and filtered. The filter cake was washed with iPrOAc and IPA and dried at a max. jacket temperature of 50° C. and reduced pressure at the rotavap to give intermediate grade title compound.

Yield (% theory): 48%

### EXAMPLE 2

### Preparation of (S)-2-((4-benzofuranyl)carbonylamino methyl)-1-((4-(2-methyl-5-(4-fluorophenyl))thiazolyl)carbonyl)piperidine—Form 1

All volumes and weights refer to the weight of Example 1.

The reactor was charged with the resulting solid (1 wt) and isopropanol (5.2 vol). The suspension was heated to get a clear solution at 82° C. The solution was cooled and seed crystals (obtained by cooling of about 1 vol % of the clear solution) was added 74° C. The resulting suspension was cooled to 10° C., stirred overnight and filtered. The filter cake was washed with IPA (0.79 vol) and dried at a maximum jacket temperature of 50° C. and reduced pressure at the rotavap. to give Form 1 as a slightly colored solid.

Yield (% theory): 96%

### EXAMPLE 3

### Micronisation of (S)-2-((4-benzofuranyl)carbonylamino methyl)-1-((4-(2-methyl-5-(4-fluorophenyl))thiazolyl)carbonyl)piperidine—Form 1

The product of Example 2 was introduced to the APTM 4″ microniser using a screw feeder. The flow rate was ranging from 15 to 24 g/minute. The microniser was set with a Venturi pressure of 8 bar and a grinding pressure of 6 bar.

Input batch size: 2000 g

Yield (g): 1850 g

Yield (%): 92%

### EXAMPLE 4

### Preparation of (S)-2-((4-benzofuranyl)carbonylamino methyl)-1-((4-(2-methyl-5-(4-fluorophenyl))thiazolyl)carbonyl)piperidine—Solvated Form

Crude Example 1 was purified by silica gel chromatography eluting with dichloromethane and methanol. The product containing fractions were combined and evaporated to dryness. The resultant product was triturated with diethyl ether to give the title product.

### EXAMPLE 5

### Preparation of (S)-2-((4-benzofuranyl)carbonylamino methyl)-1-((4-(2-methyl-5-(4-fluorophenyl))thiazolyl)carbonyl)piperidine—Form 2

2 g of Example 2 was added to 5 ml dichloromethane and temperature cycled at 0-40° C. for 4 days. The precipitate at 0° C. was isolated by filtration and evaporated to dryness to yield 895 mg of the title compound.

### EXAMPLE 6

### Preparation of (S)-2-((4-benzofuranyl)carbonylamino methyl)-1-((4-(2-methyl-5-(4-fluorophenyl))thiazolyl)carbonyl)piperidine—Form 3

400 mg of Example 4 was taken up in 2 ml of acetone. The resulting suspension was warmed in a hot water bath to 55° C. to obtain a solution. A small amount of the title product was added and the solution allowed to cool slowly. A thick precipitate was formed which was isolated by filtration and evaporated to dryness to yield 80 mg of the title compound.

### EXAMPLE 7

### Preparation of (S)-2-((4-benzofuranyl)carbonylamino methyl)-1-((4-(2-methyl-5-(4-fluorophenyl))thiazolyl)carbonyl)piperidine—Form 2

Form 1 (0.70 g) was stirred in dichloromethane (2 mL). The solid dissolved immediately and more Form 1 (0.30 g) was added. The mixture was heated at reflux temperature for 1 h. The resulting solution was cooled rapidly with stirring in an ice-water bath, a very thick slurry was obtained within 1 minute. The solid was collected by filtration (0.76 g, 76% yield). The X-Ray powder diffraction pattern was consistent with Form 2. The peak values of the Raman and infrared spectra, and the DSC trace were consistent with Form 2. The solution NMR spectra showed only a trace amount of dichloromethane.

### EXAMPLE 8

### Preparation of (S)-2-((4-benzofuranyl)carbonylamino methyl)-1-((4-(2-methyl-5-(4-fluorophenyl))thiazolyl)carbonyl)piperidine—Form 2

Form 1 (2.0 g) was stirred in dichloromethane (3 mL). The mixture was heated at reflux temperature for 30 minutes. The resulting solution was cooled rapidly with stirring in an ice-water bath, a very thick slurry was obtained within 1 minute. The solid was collected by filtration and the cake broken with a spatula. The solid was left in the filter with vacuum on for 1 h. (1.81 g, 90.5% yield). The X-Ray powder diffraction pattern was consistent with Form 2. The peak values of the Raman and infrared spectra, and the DSC trace were consistent with Form 2. The solution NMR spectra showed only a trace amount of dichloromethane.

### EXAMPLE 9

### Preparation of (S)-2-((4-benzofuranyl)carbonylamino methyl)-1-((4-(2-methyl-5-(4-fluorophenyl))thiazolyl)carbonyl)piperidine—Form 3

Form 1 (2.1 g) was stirred in acetone (15 mL). The mixture was heated at reflux temperature for 15 minutes, acetone (5 mL) was added and stirring was continued for 45 minutes. The resulting solution was cooled rapidly with stirring in an ice-water bath, a very thick slurry was obtained within 30 seconds. Stirring was continued for 10 minutes, the solid was collected by filtration and washed with cold acetone (10 mL, 5 mL). (1.16 g, 55% yield). The X-Ray powder diffraction pattern was consistent with Form 3. The peak values of the Raman and infrared spectra, and the DSC trace were consistent with Form 3. The solution NMR spectra showed no acetone.

### EXAMPLE 10

### Preparation of (S)-2-((4-benzofuranyl)carbonylamino methyl)-1-((4-(2-methyl-5-(4-fluorophenyl))thiazolyl)carbonyl)piperidine—Form 3

Form 1 (2.0 g) was stirred in acetone (15 mL). The mixture was heated at reflux temperature for 30 minutes, acetone (5 mL) was added and stirring was continued for 30 minutes. The resulting solution was cooled rapidly with stirring in an ice-water bath, a very thick slurry was obtained within 1 minute. Stirring was continued for 20 minutes, the solid was collected by filtration and washed with cold acetone (3 mL, 2 mL). (1.25 g, 62.5% yield). The X-Ray powder diffraction pattern was consistent with Form 3. The peak values of the Raman and infrared spectra, and the DSC trace were consistent with Form 3. The solution NMR spectra showed no acetone.

### EXAMPLE 11

### Preparation of (S)-2-((4-benzofuranyl)carbonylamino methyl)-1-((4-(2-methyl-5-(4-fluorophenyl))thiazolyl)carbonyl)piperidine—Form 3

Form 1 (0.20 g) was stirred in ethyl acetate (2 mL). The mixture was heated to near reflux temperature for 1 h, ethyl acetate (1.5 mL) was added and complete dissolution was achieved. The solution was cooled rapidly with stirring in a water bath, a thick slurry was obtained rapidly and ethyl acetate (1 ml) was added to ease the stirring. Stirring was continued for 30 minutes, the solid was collected by filtration. (0.09 g, 45% yield). The X-Ray powder diffraction pattern, Raman and infrared spectra, and DSC trace were consistent with Form 3. The solution NMR spectra showed only a trace amount of ethyl acetate.

### EXAMPLE 12

### Preparation of (S)-2-((4-benzofuranyl)carbonylamino methyl)-1-((4-(2-methyl-5-(4-fluorophenyl))thiazolyl)carbonyl)piperidine—Form 3

Form 1 (2.0 g) was stirred in ethyl acetate (20 mL). The mixture was heated at reflux temperature for 30 minutes, ethyl acetate (3 mL) was added and stirring was continued for 30 minutes. The resulting solution was cooled rapidly with stirring in an ice-water bath, a very thick slurry was obtained almost immediately. Stirring was continued for 20 minutes, the solid was collected by filtration and washed with ethyl acetate (2×3 mL). (1.60 g, 80% yield). The X-Ray powder diffraction pattern was consistent with Form 3. The peak values of the Raman and infrared spectra, and the DSC trace were consistent with Form 3. The TGA showed no significant mass loss till 250° C. The solution NMR spectra showed a trace amount of ethyl acetate (ca. 0.2% by weight).

### EXAMPLE 13

### Preparation of (S)-2-((4-benzofuranyl)carbonylamino methyl)-1-((4-(2-methyl-5-(4-fluorophenyl))thiazolyl)carbonyl)piperidine—Form 3

Form 1 (0.30 g) was stirred in tetrahydrofuran (1.5 mL). The mixture was heated at 80° C. until a clear solution was obtained. The solution was allowed to cool slowly to ambient temperature. The resulting thick slurry was stirred for 1 h at ambient temperature. The solid was collected by filtration and left in the filter overnight. (0.23 g, 77% yield). The X-Ray powder diffraction pattern was consistent with Form 3. The peak values of the Raman and infrared spectra, and the DSC trace were consistent with Form 3. The solution NMR spectra showed a trace amount of tetrahydrofuran (ca. 0.4% by weight).

### EXAMPLE 14

### Preparation of (S)-2-((4-benzofuranyl)carbonylamino methyl)-1-((4-(2-methyl-5-(4-fluorophenyl))thiazolyl)carbonyl)piperidine—Solvated Form

Form 3 (0.20 g from Example 9) was slurried in TBME (5 mL) and stirred at ambient temperature for 2 h. The solid was collected by filtration and washed with TBME. (Yield: 0.18 g). The X-Ray powder diffraction pattern was consistent with the solvated form. The solution NMR spectra showed circa 5.7% TBME. The TGA showed 5.71% mass loss over 35° C.-109 C° range, unresolved from 0.89% mass loss over 109° C.-161° C. range.

### EXAMPLE 15

### Preparation of (S)-2-((4-benzofuranyl)carbonylamino methyl)-1-((4-(2-methyl-5-(4-fluorophenyl))thiazolyl)carbonyl)piperidine—Solvated Form

Form 3 (0.20 g from Example 12) was slurried in TBME (5 mL) and stirred at ambient temperature for 2.5 h. TBME (3 mL) was added and stirring was continued overnight. The solid was collected by filtration and washed with TBME. (Yield: 0.16 g). The X-Ray powder diffraction pattern was consistent with solvated form. The solution NMR spectra showed circa 6.2% TBME. The TGA showed 5.92% mass loss over 34° C.-110 C° range, unresolved from 1.07% mass loss over 110° C.-175° C. range.

### EXAMPLE 16

### Preparation of (S)-2-((4-benzofuranyl)carbonylamino methyl)-1-((4-(2-methyl-5-(4-fluorophenyl))thiazolyl)carbonyl)piperidine—Solvated Form

Form 2 (0.20 g from Example 8) was slurried in TBME (5 mL) and stirred at ambient temperature for 2.5 h. The solid was collected by filtration and washed with TBME. (Yield: 0.16 g). The X-Ray powder diffraction pattern was consistent with solvated form. The solution NMR spectra showed circa 6.2% TBME. The TGA showed 5.43% mass loss over 35° C.-107 C° range, unresolved from 1.36% mass loss over 107° C.-129° C. range.

### EXAMPLE 17

### Preparation of (S)-2-((4-benzofuranyl)carbonylamino methyl)-1-((4-(2-methyl-5-(4-fluorophenyl))thiazolyl)carbonyl)piperidine—Solvated Form

Form 1 (0.50 g) was stirred in dichloromethane (1 mL). The mixture was heated at reflux temperature. The resulting clear solution was cooled rapidly with stirring in an ice-water bath, a very thick slurry was obtained within 1 minute. TBME (9 mL) was added and the slurry was stirred at ambient temperature for 2 h, then cooled in an ice-water bath and the solid was collected by filtration and washed with TBME. (Yield: 0.40 g). The X-Ray powder diffraction pattern was consistent with solvated form. The solution NMR spectra showed circa 5.1% TBME. The TGA showed 4.93% mass loss over 35° C.-111 C° range, unresolved from 0.85% mass loss over 111° C.-159° C. range.

### Characterising Data

The following characterising data were generated for a selection of the polymorphs:

### 1. NMR

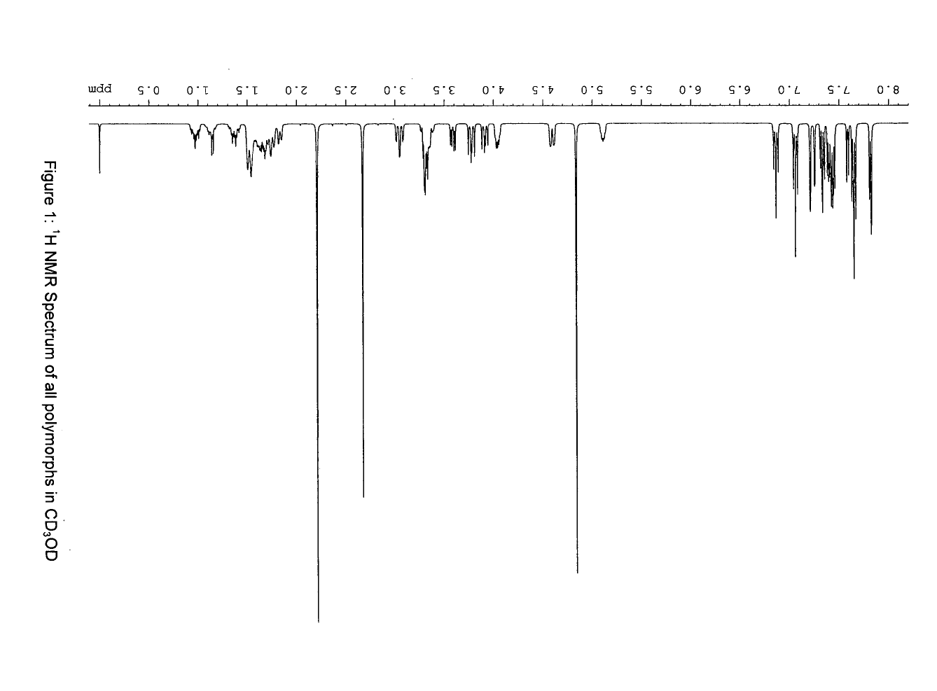

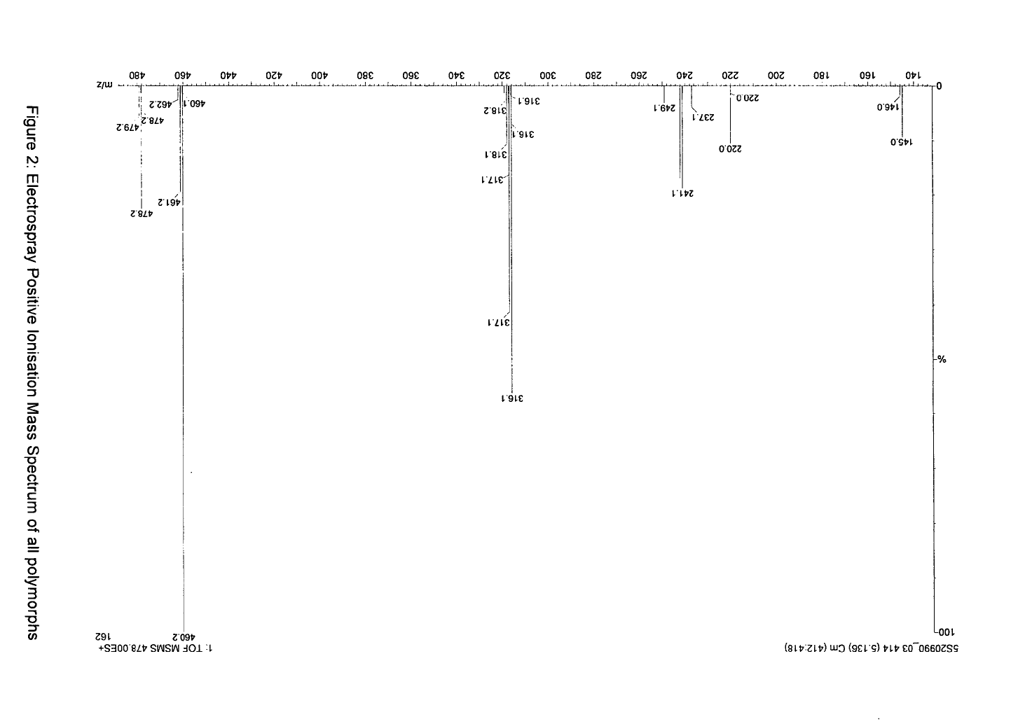

The NMR spectrum for all polymorphs is shown in FIG. 1 and has been assigned the following peaks:

¹H NMR (400 MHz, CH₃OD-d⁴) δ 0.97 (1H, m), 1.13 (1H, m), 1.37 (1H, m), 1.48-1.56 (2H, m), 1.48-1.78 (5H, m), 1.76 (1H, d), 1.83 (1H, d), 2.21 (3H, s), 2.67 (3 H, s), 3.05 (1H, td), 3.25-3.40 (3H, m), 3.59 (1H, dd), 3.78 (1H, dd), 3.91 (1H, dd), 4.04 (1H, m), 4.59 (1H, d), 5.11 (1H, m), 6.86 (2H, t), 7.06 (2H, t), 7.21 (1H, d), 7.25 (1H, d), 7.33 (1H, t), 7.34 (1H, t), 7.40 (2H, m), 7.44 (2H, m), 7.59 (1H, d), 7.65 (3H, m), 7.81 (1H, d) and 7.83 (1H, d).

### 2. Electrospray

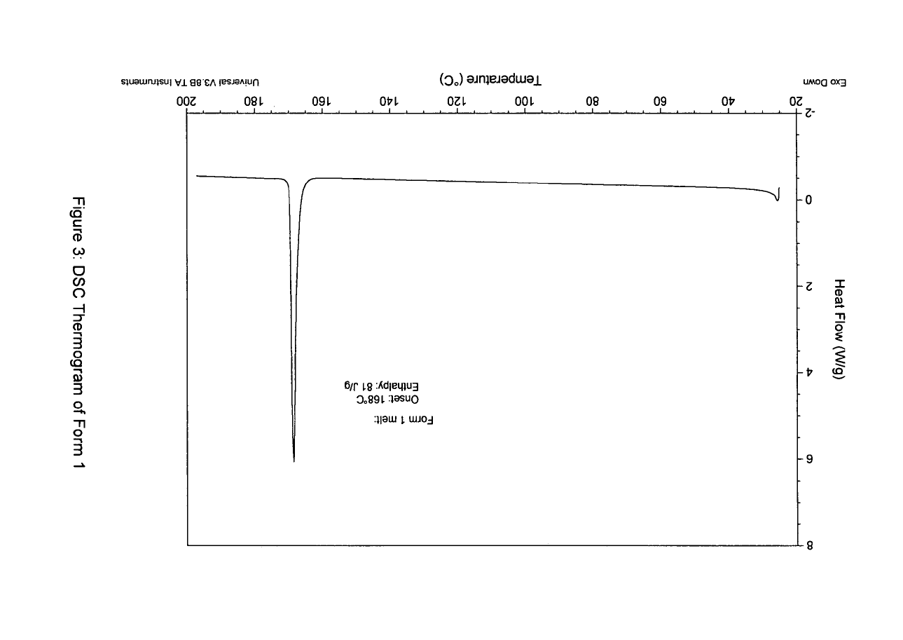

The Electrospray mass spectrum for all polymorphs is shown in FIG. 2 and has been assigned the following peaks:

MS (ES⁺) 478 (MH⁺), 460, 317, 242, 241, 220 and 145.

### 3. Thermal Analysis

The Differential Scanning Calorimetry (DSC) thermograms of Forms 1-3 were obtained using a TA Instruments calorimeter. The samples were weighed into an aluminium pan, a pan lid placed on top and lightly crimped without sealing the pan. The experiment was conducted using a heating rate of 5° C. min⁻¹ for Form 3 and 10° C. min⁻¹ for Forms 1-2. The DSC data for Forms 2 and 3 show recrystallisation upon heating to Form 1.

The results for Forms 1-3 are illustrated in FIGS. 3-5.

The DSC thermogram of the Solvated Form was obtained using a Perkin Elmer Pyris 1 calorimeter. The sample was weighed into an aluminium pan, a pan lid placed on top and lightly crimped without sealing the pan. The experiment was conducted using a heating rate of 10° C. min⁻¹.

The Solvated Form shows desolvation and crystallisation events followed by a melt consistent with Form 1. The data are illustrated in FIG. 6.

### 4. X-Ray Powder Diffraction (XRPD) Analysis

The X-ray powder diffraction (XRPD) data of Forms 1-3 are shown in FIGS. 7-9. The data were acquired on a PANalytical X.Pert Pro powder diffractometer, model PW3040/60, serial number DY1850 using an XCelerator detector. The acquisition conditions were: radiation: Cu Kα, generator tension: 40 kV, generator current: 45 mA, start angle: 2.0° 2θ, end angle: 40.0° 2 θ, step size: 0.0167° 2θ, time per step: 190.5 seconds. The samples were prepared by mounting a few milligrams of sample on a Si wafer (zero background) plates, resulting in a thin layer of powder.

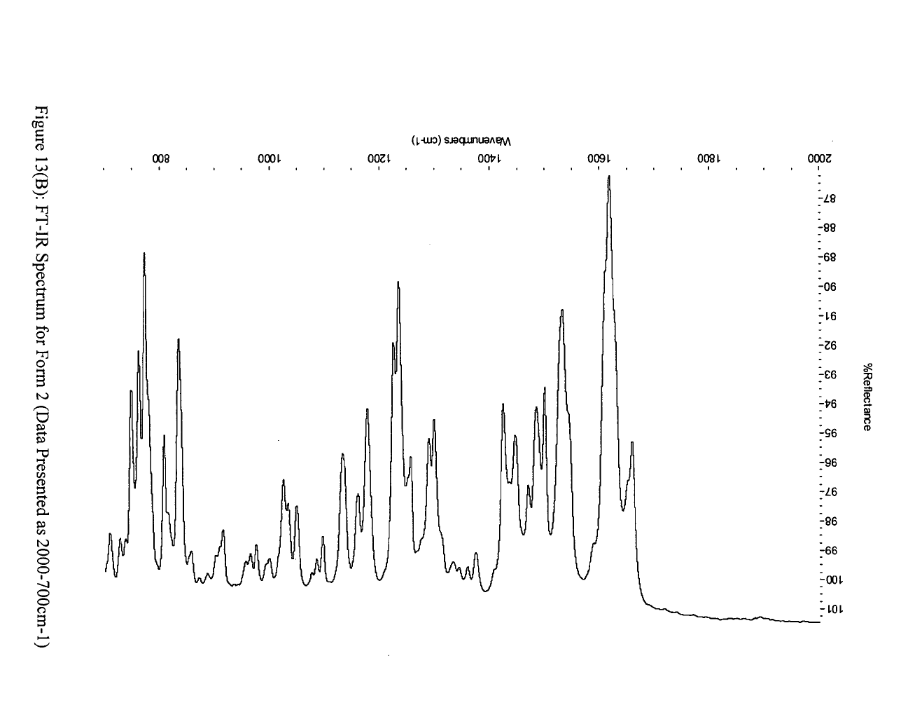

XRPD data for the Solvated Form was also acquired on a PANalytical X.Pert Pro powder diffractometer, model PW3040/60, serial number DY1850 using an XCelerator detector. The acquisition conditions for the Solvated Form were: radiation: Cu Kα, generator tension: 40 kV, generator current: 45 mA, start angle: 2.0° 2θ, end angle: 40.0° 2 θ, step size: 0.0167° 2θ, time per step: 31.750 seconds. The sample was prepared by mounting a few milligrams of sample on a Si wafer (zero background) plates, resulting in a thin layer of powder. The XRPD data for the Solvated Form is shown in FIG. 10 and an overlaid XRPD pattern of Forms 1-3 and the Solvated Form is shown in FIG. 11.

Characteristic XRPD diffraction angles (°2θ) and lattice d-spacings (\acute{Å}) of Forms 1-3 and the Solvated Form are recorded in Table 1:

TABLE 1

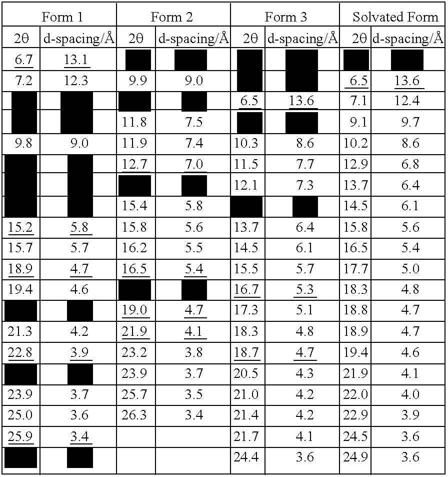

Peaks with a shaded background distinguish that Form from the other Forms. In one embodiment, the polymorphic form comprises the peaks in Table 1 with shaded backgrounds.

Other peaks (underscored and in bold) also distinguish the forms, however, there are shoulders or low intensity peaks of another form in close proximity that make these peaks less specific than those with a shaded background. In one embodiment, the polymorphic form comprises peaks in Table 1 which are underscored and in bold.

### 5. FT-IR

The FT-IR spectrum of the Forms 1-3 were recorded using a Nicolet Avatar 360 FT-IR spectrometer, serial number AEA0001623 fitted with a Diamond/ZnSe ATR Accessory at 4 cm⁻¹ resolution.

Form 1 bands were observed at:

3284, 2931, 1650, 1622, 1545, 1501, 1487, 1450, 1423, 1302, 1288, 1253, 1227, 1177, 1158, 1133, 1050, 1025, 962, 911, 852, 839, 816, 784 and 767 cm⁻¹.

Form 2 bands were observed at:

3376, 3340, 3114, 2962, 2921, 2847, 1659, 1619, 1532, 1500, 1485, 1470, 1446, 1425, 1299, 1289, 1256, 1235, 1225, 1177, 1160, 1132, 1096, 1048, 1024, 975, 914, 835, 808, 773, 762, and 748 cm⁻¹.

Form 3 bands were observed at:

3350, 3126, 2939, 2850, 1653, 1606, 1539, 1501, 1487, 1447, 1424, 1304, 1290, 1255, 1236, 1178, 1161, 1134, 1047, 1025, 974, 910, 854, 831, 806, 775, 763 and 748 cm⁻¹.

The data are illustrated in FIGS. 12-14.

### 6. FT-Raman

The Raman spectra of Forms 1-3 were recorded through a glass NMR tube using a Nicolet 960 E.S.P. FT-Raman spectrometer at 4 cm⁻¹ resolution. Excitation was achieved using a Nd:YVO4 laser (1064 nm) with a power output of 500 mW.

Form 1 bands were observed at:

3069, 2967, 2926, 1651, 1604, 1541, 1530, 1486, 1448, 1422, 1377, 1359, 1332, 1302, 1286, 1253, 1178, 1157, 1130, 1056, 899, 855, 814, 771, 673, 659, 637, 417, 358, 282, 247 and 91 cm⁻¹.

Form 2 bands were observed at:

3070, 2962, 2922, 1651, 1608, 1587, 1544, 1529, 1487, 1445, 1376, 1359, 1330, 1302, 1290, 1257, 1177, 1157, 1135, 1054, 1026, 963, 901, 814, 779, 761, 655, 638, 578, 518, 420, 357, 306, 278, 247 and 94 cm⁻¹.

Form 3 bands were observed at:

3071, 2940, 1655, 1606, 1589, 1536, 1490, 1447, 1408, 1380, 1358, 1306, 1257, 1161, 1135, 1161, 1135, 1061, 1026, 960, 902, 810, 779, 763, 653, 638, 581, 539, 417, 395, 360, 292, 246, 146 and 81 cm⁻¹.

The data are shown in FIGS. 15-17 where the x-axis shows Raman shift cm⁻¹ and the y-axis shows intensity.

1. A polymorphic form of (S)-2-((4-benzofuranyl)carbonylamino methyl)-1-((4-(2-methyl-5-(4-fluorophenyl))thiazolyl)carbonyl)piperidine characterized in that it:
i) provides an X-ray powder diffraction (XRPD) diffractogram comprising the following peaks:

| Diffraction angle | Lattice spacing |
|---|---|
| (°2θ) | (Å) |
| 8.0 | 11.1 |
| 9.1 | 9.7 |
| 13.4 | 6.6 |
| 14.1 | 6.3 |
| 18.9 | 4.7 |
| 20.2 | 4.4 |
| 23.5 | 3.8 |
| 30.6 | 2.9; |

and
ii) has an onset of melting in the range 163-173° C., as measured by DSC.

2. A polymorphic form of (S)-2-((4-benzofuranyl)carbonylamino methyl)-1-((4-(2-methyl-5-(4-fluorophenyl))thiazolyl)carbonyl)piperidine characterized in that it provides an X-ray powder diffraction pattern substantially in accordance with FIG. 7.

3. (canceled)

4. A polymorphic form of (S)-2-((4-benzofuranyl)carbonylamino methyl)-1-((4-(2-methyl-5-(4-fluorophenyl))thiazolyl)carbonyl)piperidine characterized in that it provides an FT-IR spectrum substantially in accordance with FIG. 12.

5. A polymorphic form of (S)-2-((4-benzofuranyl)carbonylamino methyl)-1-((4-(2-methyl-5-(4-fluorophenyl))thiazolyl)carbonyl)piperidine characterized in that it provides an FT-Raman spectrum substantially in accordance with FIG. 15.

6. A polymorphic form of (S)-2-((4-benzofuranyl)carbonylamino methyl)-1-((4-(2-methyl-5-(4-fluorophenyl))thiazolyl)carbonyl)piperidine characterized in that it:
i) provides X-ray powder diffraction (XRPD) diffractogram comprising the following peaks:

| Diffraction angle | Lattice spacing |
|---|---|
| (°2θ) | (Å) |
| 7.6 | 11.7 |
| 9.9 | 9.0 |
| 11.1 | 8.0 |
| 13.1 | 6.8 |
| 15.8 | 5.6 |
| 17.8 | 5.0; |

and
ii) has an onset of melting in the range 113-123° C., as measured by DSC.

7. A polymorphic form of (S)-2-((4-benzofuranyl)carbonylamino methyl)-1-((4-(2-methyl-5-(4-fluorophenyl))thiazolyl)carbonyl)piperidine characterized in that it provides an X-ray powder diffraction pattern substantially in accordance with FIG. 8.

8. (canceled)

9. A polymorphic form of (S)-2-((4-benzofuranyl)carbonylamino methyl)-1-((4-(2-methyl-5-(4-fluorophenyl))thiazolyl)carbonyl)piperidine characterized in that it provides an FT-IR spectrum substantially in accordance with FIG. 13.

10. A polymorphic form of (S)-2-((4-benzofuranyl)carbonylamino methyl)-1-((4-(2-methyl-5-(4-fluorophenyl))thiazolyl)carbonyl)piperidine characterized in that it provides an FT-Raman spectrum substantially in accordance with FIG. 16.

11-13. (canceled)

14. A polymorph according to claim 1 in isolated form.

15. A polymorph as defined in claim 1 in pure form.

16. A pharmaceutical composition comprising the polymorph as defined in claim 1 and a pharmaceutically acceptable carrier.

17-25. (canceled)

26. A pharmaceutical composition comprising the polymorph as defined in claim 6 and a pharmaceutically acceptable carrier.
